(19) United States
(12) Patent Application Publication
(10) Pub. No.: US 2016/0154011 A1
(43) Pub. Date: Jun. 2, 2016

(54) Dickkopf (DKK) Proteins as Biomarkers for Cognitive Decline Associated with Alzheimer's Disease

(71) Applicant: King's College London, London (GB)

(72) Inventor: Simon Lovestone, London (GB)

(73) Assignee: King's College London, London (GB)

(21) Appl. No.: 14/905,016

(22) PCT Filed: Jul. 14, 2014
(86) PCT No.: PCT/GB2014/052149

§ 371 Date: Jan. 14, 2016

(30) Foreign Application Priority Data
Jul. 15, 2013 (GB) 1312638.8

(51) Int. Cl. G01N 33/68

(52) U.S. Cl. CPC G01N 33/6896; G01N 2333/4704; G01N 2800/2821; G01N 2333/775; G01N 2800/52

(57) ABSTRACT

In one aspect, described herein is a method for monitoring cognitive decline in a subject, the method comprising (i) determining a level of one or more Dickkopf (Dkk) proteins in a blood sample from the subject; and (ii) comparing the level of the Dkk protein(s) to a reference value; wherein an increased level of the Dkk protein(s) in the sample compared to the reference value is indicative of increased cognitive decline in the subject.

### FIELD OF THE INVENTION

The present invention relates to the field of biomarkers of cognitive decline, including in conditions such as Alzheimer's disease. In particular, the invention relates to methods for diagnosing or predicting the progression of such conditions, especially based on biomarkers which are detectable in peripheral blood. The invention is also useful for monitoring a therapeutic treatment for cognitive decline, i.e. by providing companion biomarkers which are indicative of efficacy of the treatment regime.

### BACKGROUND

Cognitive decline is commonly associated with ageing, in many cases leading to dementia. Alzheimer's disease (AD), the most common cause of dementia in older individuals, is a debilitating neurodegenerative disease for which there is currently no cure. It destroys neurons in parts of the brain, chiefly the hippocampus, which is a region involved in coding memories. Alzheimer's disease gives rise to an irreversible progressive loss of cognitive functions and of functional autonomy. The earliest signs of AD may be mistaken for simple forgetfulness, but in those who are eventually diagnosed with the disease, these initial signs inexorably progress to more severe symptoms of mental deterioration. While the time it takes for AD to develop will vary from person to person, advanced signs include severe memory impairment, confusion, language disturbances, personality and behaviour changes, and impaired judgement. Persons with AD may become non-communicative and hostile. As the disease ends its course in profound dementia, patients are unable to care for themselves and often require institutionalisation or professional care in the home setting. While some patients may live for years after being diagnosed with AD, the average life expectancy after diagnosis is eight years.

In the past, AD could only be definitively diagnosed by brain biopsy or upon autopsy after a patient died. These methods, which demonstrate the presence of the characteristic plaque and tangle lesions in the brain, are still considered the gold standard for the pathological diagnoses of AD. However, in the clinical setting brain biopsy is rarely performed and diagnosis depends on a battery of neurological, psychometric and biochemical tests, including the measurement of biochemical markers such as the ApoE and tau proteins or the beta-amyloid peptide in cerebrospinal fluid and blood.

Better biomarkers are needed for diagnosing AD and other dementias. A biological marker that fulfils the requirements for the diagnostic test for AD would have several advantages. An ideal biological marker would be one that identifies AD cases at a very early stage of the disease, before there is degeneration observed in the brain imaging and neuropathological tests. Detection of a biomarker or panel of biomarkers could be the first indicator for starting treatment as early as possible, and also very valuable in screening the effectiveness of new therapies, particularly those that are focussed on preventing the development of neuropathological changes. A biological marker would also be useful in the follow-up of the development of the disease.

Markers related to pathological characteristics of AD, such as plaques and tangles (Aβ and tau), have been the most extensively studied. The most promising has been from studies of CSF concentration of Aβ(1-40), Aβ(1-42) and tau or the combination of both proteins in AD. Many studies have reported a decrease in Aβ(1-42) accompanied by an increase in tau in CSF.

Whilst cerebrospinal fluid (CSF) levels of Aβ and tau are promising biomarkers for diagnosis of AD they are not showing such diagnostic utility in more accessible body fluids. Cerebrospinal fluid is difficult to obtain from human patients. Its collection necessitates an invasive technique—lumbar puncture. This is a highly skilled procedure, requiring qualified and specially trained medical staff. Furthermore, it is time consuming and may require anaesthetic, as well as extended co-operation from the patient. It carries some risk including headache and is a costly procedure requiring availability of short-stay hospital beds for recovery in some cases.

In the light of the limitations of cerebrospinal fluid as a routine clinical sample, considerable interest resides in blood as a source of biomarkers for neurodegenerative conditions such as Alzheimer's disease. WO 06/035237 describes proteomics studies that identified a number of differentially expressed proteins and described certain methods for the diagnosis of Alzheimer's disease. WO 2010/084327 describes protein biomarkers in plasma which are useful for diagnosing Alzheimer's disease.

However, it remains the case that biomarkers known in the art to be associated with cognitive decline have had limited or insignificant prognostic value. Whilst current clinical diagnosis of Alzheimer's disease based on general neurological symptoms and imprecise cognitive function tests is reasonably robust, it remains a problem to describe, and in particular to predict, the likely progress of disease in living patients. Thus, prognosis, as well as diagnosis, remains a problem in the art in connection with living patients. The present invention seeks to overcome problems associated with the prior art.

### SUMMARY OF THE INVENTION

In one aspect, the present invention provides a method for monitoring cognitive decline in a subject, the method comprising (i) determining a level of one or more Dickkopf (Dkk) proteins in a blood sample from the subject; and (ii) comparing the level of the Dkk protein(s) to a reference value; wherein an increased level of the Dkk protein(s) in the sample compared to the reference value is indicative of increased cognitive decline in the subject.

In one embodiment, the blood sample comprises blood plasma or serum.

In further embodiments, the Dkk protein comprises Dkk1, Dkk3, Dkk4 and/or DkkL1 (soggy1).

In another embodiment, the method further comprises determining a level of clusterin in the sample, wherein an increased level of clusterin in the sample compared to the reference value is indicative of increased cognitive decline in the subject.

In one embodiment, the method further comprises determining a level of one or more additional biomarkers in the sample, wherein the additional biomarker is selected from a plasma protein as defined in any of Tables 3 to 10.

In one embodiment, the cognitive decline is associated with Alzheimer's disease.

In another embodiment, the reference value comprises a level of the Dkk protein in a sample from a healthy subject.

In a further aspect, the invention provides a method for monitoring the efficacy of a therapeutic treatment for cognitive decline, comprising (i) monitoring cognitive decline in the subject by a method as defined above; and (ii) repeating the monitoring one or more times following administration of the treatment to the subject; wherein a decreased level of the Dkk protein(s) in the sample following administration of the treatment is indicative of therapeutic efficacy of the treatment.

In another aspect, the invention provides a method for treating cognitive decline in a subject, the method comprising (i) administering a therapeutic treatment for cognitive decline to the subject; (ii) monitoring cognitive decline in the subject by a method as defined above, wherein the monitoring is performed before and after administration of the treatment to the subject; and (iii) providing further treatment to the subject based on the results of the monitoring.

In one embodiment, if the monitoring indicates a decreased level of the Dkk protein(s) in the sample following administration of the treatment, the further treatment comprises continuing the therapeutic treatment defined in step (i).

In another embodiment, if the monitoring indicates an increased level of the Dkk protein(s) in the sample following administration of the treatment, the further treatment comprises (a) increasing a dose of the therapeutic treatment defined in step (i); and/or (b) administering an alternative therapeutic treatment to the subject, wherein the alternative therapeutic treatment is different to the therapeutic treatment defined in step (i).

In a further aspect, the invention provides a therapeutic agent for use in treating cognitive decline in a subject, wherein the subject has been monitored for cognitive decline by a method as defined above.

In a further aspect, the invention provides a kit for monitoring cognitive decline in a subject, the kit comprising one or more reagents suitable for detecting one or more Dickkopf (Dkk) proteins in a blood sample from the subject.

In one embodiment, the kit comprises one or more antibodies which bind to one or more Dkk proteins.

In another embodiment, the kit comprises an ELISA assay for one or more Dkk proteins.

### BRIEF DESCRIPTION OF THE FIGURES

FIG. 1A shows clustering of plasma proteins based on Spearman's correlation. Dkk4 and Dkk1 have overlapped. FIG. 1B shows clustering of plasma proteins based on Spearman's partial correlation.

### DETAILED DESCRIPTION OF THE INVENTION

Monitoring Cognitive Decline

The present invention provides in one aspect a method for monitoring cognitive decline in a subject. Monitoring may include various diagnostic and prognostic applications related to the assessment of cognitive decline in the subject. Thus in particular embodiments, the method may comprise (i) measuring cognitive decline; (ii) determining a level of cognitive decline; (iii) predicting a risk of cognitive decline; (iv) determining or predicting a rate of progression of cognitive decline; (v) diagnosing cognitive decline; and/or (vi) predicting the onset of cognitive decline.

Cognitive decline is typically a progressive impairment in cognitive function, which is commonly associated with old age. Thus in embodiments of the present invention, the cognitive decline may be age-related. In a preferred embodiment, the cognitive decline is associated with Alzheimer's disease or other forms of age-related dementia. Thus the present invention may be used to monitor Alzheimer's disease, and in various diagnostic and prognostic applications associated with this condition (as described above with reference to cognitive decline).

In a particularly preferred embodiment, the method may be used to determine or predict the rate of cognitive decline associated with Alzheimer's disease. In another preferred embodiment, the method is used to determine or predict the rate of conversion of mild cognitive impairment (MCI) to Alzheimer's disease. MCI is defined as a significant cognitive impairment in the absence of dementia, for instance involving some memory loss and other changes without losing the ability to function independently.

In some embodiments, the method may be used to determine whether a subject suffering from cognitive decline is suffering from Alzheimer's disease. For instance, the method may be used to determine a level of activity of the Aβ/clusterin/Dkk pathway in the subject, which may be indicative of the development of Alzheimer's disease. Thus in these embodiments, the method may be used to distinguish AD from cognitive decline associated with other conditions.

Subject

Typically the subject is a human. In a preferred embodiment the subject is an adult human, more preferably an elderly subject, e.g. 50 years or older, 60 years or older, 65 years or older, 70 years or older, 75 years or older, or 80 years or older.

The subject is typically suspected to be suffering from cognitive decline. For instance, the subject may show one or more symptoms of cognitive decline, such as memory loss, confusion, inability to concentrate or perform daily tasks. In some embodiments, the subject may already be diagnosed with a form of cognitive decline (e.g. MCI), and the method may be used to predict (the rate of) progression of the condition (e.g. to AD).

Determining a Level of Dickkopf (Dkk) Proteins

In embodiments of the present invention, the level of one or more Dkk proteins in the sample is determined. Dickkopf (Dkk) proteins are the products of an evolutionary conserved small gene family of four members (Dkk1-4) and a unique Dkk3-related gene, Dkkl1 (soggy). The secreted proteins typically antagonize Wnt/beta-catenin signaling, by inhibiting the Wnt coreceptors Lrp5 and 6. Additionally, Dkks are high affinity ligands for the transmembrane proteins Kremen1 and 2, which also modulate Wnt signaling. Dkks play an important role in vertebrate development, where they locally inhibit Wnt regulated processes such as antero-posterior axial patterning, limb development, somitogenesis and eye formation. In the adult, Dkks are implicated in bone formation and bone disease and cancer amongst other conditions (see Niehrs C., Function and biological roles of the Dickkopf family of Wnt modulators, Oncogene 2006, 25(57):7469-81).

Amino acid and nucleotide sequences of the human Dkk proteins are available from publicly available databases, e.g. as shown in the following table:

| | Dkk1 | Dkk2 | Dkk3 | Dkk4 | DkkL1 (soggy1) |
|---|---|---|---|---|---|
| Entrez | 22943 | 27123 | 27122 | 27121 | 27120 |
| Ensembl | ENSG00000107984 | ENSG00000155011 | ENSG00000050165 | ENSG00000104371 | ENSG00000104901 |
| UniProt | O94907 | Q9UBU2 | Q9UBP4 | Q9UBT3 | Q9UK85 |
| RefSeq | NM_012242.1 | NM_014421.1 | NM_001018057.1 | NM_014420.2 | NM_001197301.1 |
| (mRNA) | | | | | |
| RefSeq | NP_036374.1 | NP_055236.1 | NP_001018067.1 | NP_055235.1 | NP_001184230.1 |
| (protein) | | | | | |

In particular embodiments of the present invention, the Dkk protein is selected from Dkk1, Dkk2, Dkk3, Dkk4 and DkkL1 (soggy1). More preferably, the Dkk protein is selected from Dkk1, Dkk3, Dkk4 and DkkL1 (soggy1).

For instance, in one preferred embodiment, the Dkk protein is Dkk1. In another preferred embodiment, the Dkk protein is Dkk3. In another preferred embodiment, the Dkk protein is Dkk4. In another preferred embodiment, the Dkk protein is DkkL1 (soggy1).

The level of the Dkk protein(s) in the sample may be determined by any suitable method. For example, methods for detecting protein biomarkers may include the use of an antibody, capture molecule, receptor, or fragment thereof which selectively binds to the protein. Antibodies which bind to the biomarkers described herein are known or may be produced by methods known in the art, including immunization of an animal and collection of serum (to produce polyclonal antibodies) or spleen cells (to produce hybridomas by fusion with immortalised cell lines leading to monoclonal antibodies). Detection molecules such as antibodies may optionally be bound to a solid support such as, for example, a plastic surface or beads or in an array. Suitable test formats for detecting protein levels include, but are not limited to, an immunoassay such as an enzyme-linked immunosorbent assay (ELISA), radioimmunoassay (RIA), Western blotting and immunoprecipitation.

Alternatively the level of the Dkk protein may be determined by mass spectroscopy. Mass spectroscopy allows detection and quantification of an analyte by virtue of its molecular weight. Any suitable ionization method in the field of mass spectroscopy known in the art can be employed, including but not limited to electron impact (El), chemical ionization (CI), field ionization (FDI), electrospray ionization (ESI), laser desorption ionization (LDI), matrix assisted laser desorption ionization (MALDI) and surface enhanced laser desorption ionization (SELDI). Any suitable mass spectrometry detection method may be employed, for example quadrapole mass spectroscopy (QMS), fourier transform mass spectroscopy (FT-MS) and time-of-flight mass spectroscopy (TOF-MS).

Sample

The sample used in the present method is preferably derived from blood. Suitable sample types include whole blood or any fractions of blood, including fractions which are cell free. Preferably the sample comprises blood plasma or blood serum. Suitable methods for obtaining and fractionating blood samples are well known to those skilled in the art.

Comparison to Reference Value

In the present method, the level of Dkk protein(s) in the sample from the test subject is compared to a reference value. The reference value may be, for example, a predetermined measurement of a level of Dkk protein(s) which is indicative of a particular level of cognitive decline. For instance the reference value may be a control value which is indicative of a normal (healthy) level of Dkk proteins, or a value which is indicative of mild cognitive impairment.

In one embodiment, the reference value is a level of the Dkk protein in a reference sample, which may be obtained from a subject who is not suffering from or suspected of suffering from cognitive decline (e.g. AD). For instance the reference sample may be from a healthy subject. The reference sample may be processed and analysed in the same manner as the test sample. The reference sample or value may be gender-matched and/or age-matched, more suitably matched for genetic or ethnic background or other such criteria as are routinely applied in matching of clinical samples to controls, insofar as the levels of the relevant biomarker in plasma are dependent on such factors. In some embodiments the reference sample may be an earlier sample taken from the same subject before the onset of cognitive decline, e.g. before symptoms of Alzheimer's disease are apparent.

Typically an increase in the level of the Dkk protein in the test sample compared to the reference sample is indicative of increased cognitive decline in the subject. For instance, an increase in the level of the Dkk protein compared to a control value of the Dkk protein in a healthy control may indicate that the subject has developed, or is likely to develop Alzheimer's disease. Similarly an increase in the level of the Dkk protein in the subject compared to a level from the same subject at an earlier date may indicate that the subject's cognitive function has declined since the earlier date, and/or is likely to decline in the near future. By regular measurement of Dkk protein levels in this way, the method may be used to monitor and/or predict, for instance, the rate of cognitive decline and/or the progression of subjects from MCI to AD.

Biomarker Combinations

In embodiments of the present invention, the levels of one or more Dkk proteins are used as biomarkers of cognitive decline, e.g. in the diagnosis and prognosis of AD. In particular embodiments, the method may comprise determining the levels of:

(i) Dkk1, Dkk3, Dkk4 and DkkL1 (soggy1);

(ii) Dkk1, Dkk3 and Dkk4;

(iii) Dkk1, Dkk3, and DkkL1 (soggy1);

(iv) Dkk1, Dkk4 and DkkL1 (soggy1);

(v) Dkk3, Dkk4 and DkkL1 (soggy1);

(vi) Dkk1 and Dkk3;

(vii) Dkk1 and Dkk4;

(viii) Dkk1 and DkkL1 (soggy1);

(ix) Dkk3 and Dkk4;

(x) Dkk3 and DkkL1 (soggy1);

(xi) Dkk4 and DkkL1 (soggy1).

In further embodiments, the determination of one or more Dkk proteins as described above may be performed in combination with the measurement of one or more further biomarkers of cognitive decline. For instance, in one embodiment the method further comprises determining a level of clusterin in the sample. The level of clusterin may then be compared to a reference value. The reference value may correspond to the same type of value as described above in relation to Dkk proteins, e.g. a level of clusterin in a reference sample from a healthy individual or from the same subject before the onset of dementia. An increased level of clusterin in the test sample compared to the reference value is typically indicative of increased cognitive decline in the subject. Amino acid and nucleotide sequences of human clusterin are available from public databases, e.g. Entrez 1191; Ensembl ENSG00000120885; UniProt P10909; RefSeq (mRNA) NM_001831; and RefSeq (protein) NP_001822).

Moreover, as shown below in the Examples the levels of Dkk and clusterin proteins in plasma correlate with a number of further plasma proteins. In some embodiments, these further plasma proteins can also be used as biomarkers of cognitive decline. For instance, suitable further biomarkers include (i) one or more plasma proteins as defined in Table 3, which correlate with clusterin levels in plasma; (ii) one or more plasma proteins as defined in Table 4, which correlate with Dkk1 levels in plasma; (iii) one or more plasma proteins as defined in Table 5, which correlate with Dkk3 levels in plasma; (iv) one or more plasma proteins as defined in Table 6, which correlate with Dkk4 levels in plasma; and/or (v) one or more plasma proteins as defined in Table 7, which correlate with DkkL1 (soggy1) levels in plasma.

In preferred embodiments, levels of the further biomarkers correlate with both clusterin and at least one Dkk protein (e.g. Dkk1 and/or Dkk4). For instance, the further biomarkers used in the method may comprise a plasma protein as defined in any of Tables 8, 9, 10 and 12. In particularly preferred embodiments, the method further comprised determining a level of complement C5 (UniProt P01031, Entrez 727) and/or Muellerian-inhibiting substance (MIS, also known as anti-Muellerian hormone, UniProt P03971, Entrez 268) in the sample. Levels of these additional biomarkers may be compared to reference values as described above in relation to Dick proteins. Typically an increase in the additional biomarkers (e.g. complement C5 and/or MIS) is indicative of increased cognitive decline in the subject.

Monitoring Efficacy of a Therapeutic Treatment

In embodiments of the present invention, the present method may be used in order to monitor the efficacy of a therapeutic treatment for cognitive decline. For instance, levels of Dkk protein(s) and optionally clusterin and/or one or more additional biomarkers as described above may be determined before and after administration of the therapeutic treatment. Thus Dkk proteins may be used in embodiments of the present invention as companion diagnostic biomarkers.

Typically decreased levels of the Dkk protein(s) (and optionally clusterin and/or one or more additional biomarkers as described above) is indicative of therapeutic efficacy, particularly where the levels are compared against a previous level of Dkk proteins in the same subject. In some embodiments, no change in levels of the Dkk protein(s) may be indicative of a therapeutic effect, particularly e.g. if the levels are compared to those of a healthy subject or where the levels of Dkk proteins are no longer increasing in the subject (i.e. where the levels of Dkk proteins were previously increasing in a subject, indicating cognitive decline, and the therapeutic treatment has prevented a further increase).

If levels of the Dkk protein(s) increase in the subject, i.e. when compared to previous levels in the same subject, this may indicate a lack of efficacy of the treatment and a need to devise an alternative therapeutic strategy. In this instance, in particular embodiments the subject may be switched to a different therapeutic agent or the dose of the current agent increased.

Treatments for Alzheimer's disease are known in the art. For instance, in particular embodiments, the therapeutic treatment may be an acetylcholinesterase inhibitor (e.g. tacrine, rivastigmine, galantamine or donepezil). In an alternative embodiment, the therapeutic treatment may be an NMDA receptor antagonist (e.g. memantine). Many novel therapies for Alzheimer's disease are currently in development, and may be used in combination with embodiments of the present invention. For instance, in one embodiment, the treatment may be a biopharmaceutical agent such as an antibody, e.g. an antibody which binds to a beta amyloid (Aβ) peptide such as Aβ 1-40 or Aβ 1-42. In some embodiments, the method of the present invention may be used to establish or confirm the efficacy of such novel treatments. Alternatively, such novel therapies may be administered to subjects who show no response to more traditional treatment regimes.

In one preferred embodiment, the therapeutic agent targets a pathway associated with Dkk proteins. For instance the therapeutic agent may bind to or otherwise inhibit a target within the wnt pathway (see e.g. KiHick et al. Clusterin regulates beta-amyloid toxicity via Dickkopf-1-driven induction of the wnt-PCP-JNK pathway, Molecular Psychiatry 2012: 1-11). The therapeutic agent may, for instance, bind to or inhibit expression of a target such as β-amyloid, clusterin or a Dkk protein, or a downstream component of this pathway. Antibodies and/or small molecule inhibitors against such targets are known or may be generated using methods known in the art.

The therapeutic agent may be administered to a subject using a variety of techniques. For example, the agent may be administered systemically, which includes by injection including intramuscularly or intravenously, orally, sublingually, transdermally, subcutaneously, internasally. The concentration and amount of the therapeutic agent to be administered will typically vary, depending on the type and severity of cognitive decline, the type of agent that is administered, the mode of administration, and the age and health of the subject.

The therapeutic agent may be formulated in a pharmaceutical composition in e.g. solid or tablet form or in liquid form, e.g. together with a pharmaceutically acceptable diluent. The compositions may routinely contain pharmaceutically acceptable amounts of diluents, excipients and other suitable carriers. Appropriate carriers and formulations are described, for example, in Remington's Pharmaceutical Sciences (Remington's Pharmaceutical Sciences, Mack Publishing Company, Easton, Pa., USA 1985).

Kits

In further embodiments, the present invention provides a kit suitable for performing the method as described above. In particular, the kit may comprise reagents suitable for detecting the biomarkers described above, e.g. one or more Dkk proteins and optionally clusterin and/or one or more additional biomarkers as described above. Typically the reagents may comprise antibodies which bind specifically to the biomarkers. For instance the kit may comprise one, two, three or four different antibodies, each of which binds to a different biomarker selected from those defined above.

Such kits may optionally further comprise one or more additional components, particularly reagents suitable for performing an ELISA assay using antibodies which bind to the biomarkers. For instance, the kits may comprise capture and detection antibodies for each biomarker, secondary antibodies, detection reagents, solid phases (e.g. reaction plates or beads), standards (e.g. known concentrations of each biomarker in the form of recombinant proteins) as well as buffers suitable for performing any of step of an ELISA method. The kits may further comprise vials, containers and other packaging materials for storing the above reagents, as well as instructions for performing a method as defined herein.

The invention will now be described by way of example only with reference to the following non-limiting embodiments.

### EXAMPLES

Peripheral Signatures of the Ab-Clusterin-Dkk Neurotoxicity Pathway as Blood Based Biomarkers

Previously we and others have identified a molecular pathway responsible for the neurotoxic signal of Aβ (see Killick R, et al. Clusterin regulates beta-amyloid toxicity via Dickkopf-1-driven induction of the wnt-PCP-JNK pathway, Mol Psychiatry 2012; Rosi M C, et al. Increased Dickkopf-1 expression in transgenic mouse models of neurodegenerative disease, Journal Of Neurochemistry 2010; 112(6):1539-51; Cappuccio I, et al. Induction of Dickkopf-1, a negative modulator of the Wnt pathway, is required for the development of ischemic neuronal death, J Neurosci. 2005; 25(10):2647-57; and Purro S A, et al. The Secreted Wnt Antagonist Dickkopf-1 Is Required for Amyloid beta-Mediated Synaptic Loss, The Journal of neuroscience: the official journal of the Society for Neuroscience 2012; 32(10):3492-8).

This pathway includes the Wnt modifier Dkk1 and the AD risk gene clusterin, and is based on the findings that:
Aβ induces Dkk1 expression in cells and animal models;
Suppression of Dkk1 by siRNA prevents Aβ toxicity
Increased Dkk1 in transgenic mice induces tau phosphorylation and cognitive deficits;
In neuronal cultures, Aβ induces changes in clusterin trafficking;
Aβ induced Dkk1 expression and neuronal toxicity is prevented by siRNA knockdown of clusterin;
The Aβ induced, clusterin mediated, increase in Dkk1 expression induces a cascade of events, most likely involving wnt-PCP and resulting in the increased expression of a series of transcription factors (TFs);
The Ab-clu-dkk1-wntPCP-TF pathway is detectable in animal models and in disease brain in man in a myloidopathy but not tauopathy.

In the present study, it was investigated whether elements of this pathway are detectable as proteins in blood and can be used as biomarkers of cognitive decline.

Methods

We utilised an extensive aptamer capture array technology (somaMERs; SomaLogic, Boulder, Colo.; see Gold L, et al. Aptamer-based multiplexed proteomic technology for biomarker discovery, PLoS One 2010; 5(12):e15004) to determine protein concentration first of the key proteins clusterin and Dkk isoforms (Dkk1, Dkk3, Dkk4, DkkL1 (Soggy1)) and then to correlate patterns of protein expression with these pathway initiators.

We analysed 707 plasma samples from the AddNeuroMed study and the KHP Dementia Cohorts.

TABLE 1

| | Non-dementia | MCI non | MCI | Alzheimer's |
|---|---|---|---|---|
| | controls | convertors | convertors | disease |
| Number | 209 | 106 | 43 | 319 |
| Age (Median | 76 (7) | 77 (10) | 76 (9) | 79 (10) |
| (IQR)) | | | | |
| Gender (M/F) | 102/107 | 40/66 | 17/26 | 98/221 |
| Baseline MMSE | 29 (1.0) | 27 (2.0) | 26.5 (3.0) | 20.0 (7.0) |
| (median) | | | | |
| APOE genotype | 153/51/5 | 73/29/4 | 17/23/3 | 139/136/44 |
| (e4: 0/1/2) | | | | |

Results

1. Hypothesis driven analysis showed a highly significant correlation of DKKL1, DKK3 and DKK4 with [Clusterin]ₚₗₐₛₘₐ

TABLE 2

| | Correlation to clusterin | | |
|---|---|---|---|
| | Correlation | p-value | FDR p-value |
| DkkL1 (soggy1) | 0.19 | 2.70E−07 | 1.09E−06 |
| DKK1 | 0.05 | 0.212 | 0.212 |
| Dkk3 | 0.101 | 0.007 | 0.009 |
| Dkk4 | 0.107 | 0.004 | 0.008 |

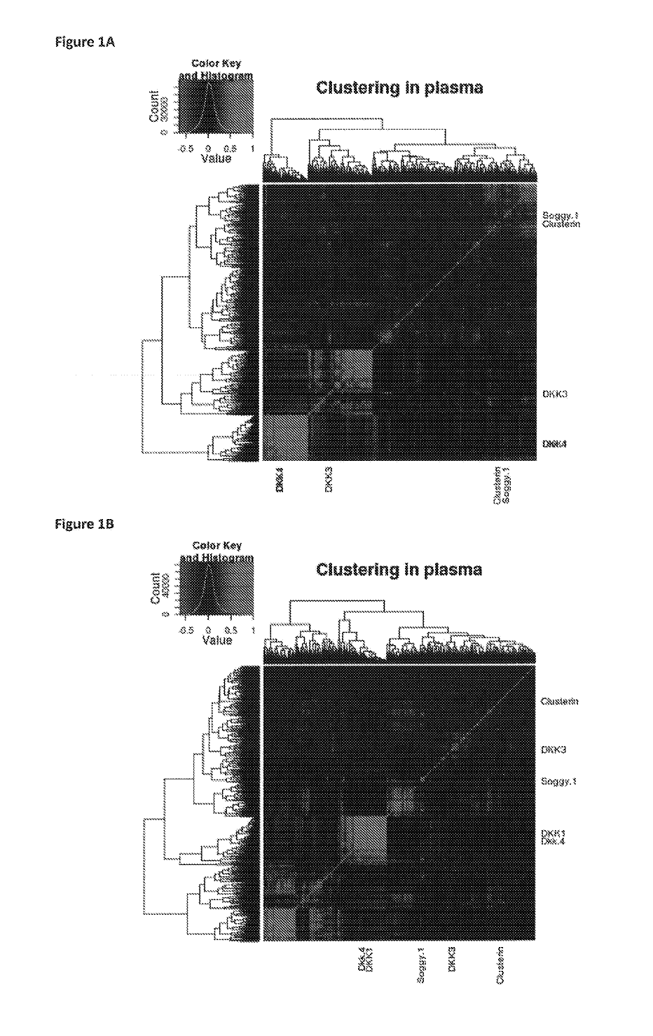

2. Clustering of plasma proteins shows modules of gene expression correlating with plasma Dkk1/4, DKK3 and DkkL1(soggy1)/clusterin (see FIG. 1A and FIG. 1B, and Tables 3 to 7 below).

TABLE 3
Clusterin to plasma protein correlations

| Protein | GeneName | Uniprot | Entrez | Spearmans | P-value | BH MTC |
|---|---|---|---|---|---|---|
| C1OBP | C1QBP | Q07021 | 708 | 0.442040251640034 | 1.39E−37 | 1.41E−34 |
| NG36 | EHMT2 | Q96KQ7 | 10919 | 0.425044091342166 | 2.79E−34 | 1.41E−31 |
| I.309 | CCL1 | P22362 | 6346 | 0.37921614306371 | 1.64E−26 | 5.54E−24 |
| C6 | C6 | P13671 | 729 | 0.347969118410633 | 4.90E−22 | 1.24E−19 |
| C5b..6.Complex | C5 | P01031 | 727 | 0.335570286337893 | 2.01E−20 | 4.06E−18 |
| C1.Esterase.Inhibitor | SERPING1 | P05155 | 710 | 0.334571331597439 | 2.69E−20 | 4.53E−18 |
| IL.34 | IL34 | Q6ZMJ4 | 146433 | 0.322058753258257 | 9.17E−19 | 1.32E−16 |
| ERBB1 | EGFR | P00533 | 1956 | 0.292759910783998 | 1.71E−15 | 2.16E−13 |
| Apo.D | APOD | P05090 | 347 | 0.290908514528206 | 2.67E−15 | 2.71E−13 |
| ERBB3 | ERBB3 | P21860 | 2065 | 0.290882392287162 | 2.68E−15 | 2.71E−13 |

Table above shows top 10 spearman's partial correlations of clusterin to plasma proteins. Significance based on BH MTC<0.05 and |spearman's partial correlation|>0.25. Significant=38 and Insignificant correlations=973.

TABLE 4
Dkk1 to plasma protein correlations

| Protein | GeneName | Uniprot | Entrez | Spearmans | P-value | BH MTC |
|---|---|---|---|---|---|---|
| RANTES | CCL5 | P13501 | 6352 | 0.78652013919164 | 1.92E−240 | 1.94E−238 |
| RAN | RAM | P62626 | 5901 | 0.774426446774686 | 2.84E−222 | 2.61E−220 |
| GPVI | GP6 | Q9HCN6 | 51206 | 0.76505585001155 | 1.67E−209 | 1.41E−207 |
| P.Selectin | SELP | P16109 | 6403 | 0.741657080299886 | 9.79E−182 | 7.62E−180 |
| TIMP.3 | TIMP3 | P35625 | 7078 | 0.73322917907039 | 5.92E−173 | 4.27E−171 |
| TCTP | TPT1 | P13693 | 7178 | 0.73094031088223 | 1.15E−170 | 7.73E−169 |
| CLC1B | CLEC1B | Q9P126 | 51266 | 0.73077713073219 | 1.66E−170 | 1.05E−168 |
| DRG.1 | VTA1 | Q9NP79 | 51534 | 0.729865034060067 | 1.32E−169 | 7.85E−168 |
| Midkine | MDK | P21741 | 4192 | 0.72624838252127 | 4.22E−166 | 2.37E−164 |
| Anglopoletin.1 | ANGPT1 | Q15389 | 284 | 0.72589095322101 | 9.27E−166 | 4.93E−164 |

Table above shows top 10 spearman's partial correlations of DKK1 to plasma proteins. Significance based on BH MTC<0.05 and |spearman's partial correlation|>0.25. Significant=227 and Insignificant correlations=784.

TABLE 5
Dkk3 to plasma protein correlations

| Protein | GeneName | Uniprot | Entrez | Spearmans | P-value | BH MTC |
|---|---|---|---|---|---|---|
| RGMB | RGMB | Q6NW40 | 285704 | 0.535667027056584 | 4.11E−61 | 4.15E−58 |
| Osteoblast.specif.transer.fact.2 | RUNX2 | Q13950 | 860 | 0.491331207185669 | 1.05E−48 | 5.30E−46 |
| ROR1 | ROR1 | Q01973 | 4919 | 0.426177769889113 | 1.71E−34 | 5.76E−32 |
| TNF.sR.I | TNFRSF1A | P19438 | 7132 | 0.407925783450307 | 3.40E−31 | 8.58E−29 |
| WFKN2 | WFIKKN2 | Q8TEU8 | 124857 | 0.403466649654001 | 1.98E−30 | 4.01E−28 |
| LSAMP | LSAMP | Q13449 | 4045 | 0.36726741698695 | 9.98E−25 | 1.68E−22 |
| MATN2 | MATN2 | ODD339 | 4147 | 0.365396448664378 | 1.86E−24 | 2.69E−22 |
| Spondin.1 | SPON1 | Q9HCB6 | 10418 | 0.359575563705289 | 1.26E−23 | 1.49E−21 |
| CNTFR.alpha | CNTFR | P26992 | 1271 | 0.359413963570335 | 1.32E−23 | 1.48E−21 |
| BMP.6 | BMP6 | P22004 | 654 | 0.356671810270882 | 3.00E−23 | 2.97E−21 |

Table above shows top 10 spearman's partial correlations of DKK3 to plasma proteins. Significance based on BH MTC<0.05 and |spearman's partial correlation|>0.25. Significant=69 and Insignificant correlations=942.

TABLE 6
Dkk4 to plasma protein correlations

| Protein | GeneName | Uniprot | Entrez | Spearmans | P-value | BH MTC |
|---|---|---|---|---|---|---|
| PDGF.BB | PDGFB | P01127 | 5155 | 0.809258311361563 | 6.04E−281 | 1.02E−278 |
| Protease.nexin.I | SERPINE2 | P07093 | 5270 | 0.794881954480216 | 2.80E−254 | 4.04E−252 |
| ON | SPARC | P09486 | 6678 | 0.793616238619912 | 4.12E−252 | 5.20E−250 |
| Thrombospondin.1 | THBS1 | P07996 | 7057 | 0.753455446898589 | 4.61E−195 | 5.17E−193 |
| RAN | RAM | P62826 | 5901 | 0.727642831156493 | 1.93E−167 | 1.95E−165 |
| RANTES | CCL5 | P13501 | 6352 | 0.7133309666146 | 2.53E−154 | 2.32E−152 |
| GPVI | GP6 | Q9HCN6 | 51206 | 0.706973033800932 | 6.40E−149 | 5.39E−147 |
| Midkine | MDK | P21741 | 4192 | 0.697063522673885 | 5.76E−141 | 4.48E−139 |
| TCTP | TPT1 | P13693 | 7178 | 0.682863410631375 | 1.82E−130 | 1.32E−128 |
| CLC1B | CLEC1B | Q9P126 | 51266 | 0.681899083897281 | 8.68E−130 | 5.85E−128 |

Table above shows top 10 spearman's partial correlations of Dkk4 to plasma proteins. Significance based on BH MTC<0.05 and |spearman's partial correlation|>0.25. Significant=253 and Insignificant correlations=758.

TABLE 7
DkkL1 (Soggy1) to plasma protein correlations

| Protein | GeneName | Uniprot | Entrez | Spearmans | P-value | BH MTC |
|---|---|---|---|---|---|---|
| kallikrein.14 | KLK14 | Q9P0G3 | 43847 | 0.559874150150244 | 5.08E−69 | 5.11E−66 |
| Apo.D | APOD | P05090 | 347 | 0.504665525783042 | 3.60E−52 | 1.82E−49 |
| Kallikrein.6 | KLK6 | Q92876 | 5653 | 0.49867980532052 | 1.37E−50 | 4.63E−48 |
| sRANKL | TNFSF11 | O14788 | 8600 | 0.493479065898525 | 3.00E−49 | 7.56E−47 |
| Cytidylate.kinase | CMPK1 | P30085 | 51727 | 0.479641947661681 | 7.70E−46 | 1.56E−43 |
| IL.5 | IL5 | P05113 | 3567 | 0.448655991247157 | 6.15E−39 | 8.88E−37 |
| PKC.G | PRKCG | P05129 | 5582 | 0.446518780386113 | 1.70E−38 | 2.15E−36 |
| OSM | OSM | P13725 | 5008 | 0.44085263315561 | 2.41E−37 | 2.71E−35 |
| Carbonic.Anhydrase.IV | CA4 | P22748 | 762 | 0.427921598960899 | 8.00E−35 | 8.09E−33 |
| ARGI1 | ARG1 | P05089 | 383 | 0.414221164260975 | 2.65E−32 | 2.23E−30 |

Table above shows top 10 spearman's partial correlations of Soggy1 to plasma proteins. Significance based on BH MTC<0.05 and |spearman's partial correlations|>0.25. Significant=162 and Insignificant correlations=859.

3. Plasma proteins correlating with [Clusterin]ₚₗₐₛₘₐ show overlap with those correlating with Dkk1, Dkk4 and DkkL1.

TABLE 8
Overlap between plasma proteins which correlate with both clusterin and Dkk1

| | | | | CLU | | | DKK1 | | |
|---|---|---|---|---|---|---|---|---|---|
| Protein | GeneName | Uniprot | Entrez | Spearmans | P.value | BH.MTC | Spearmans | P.value | BH.MTC |
| C5b..6.Complex | C5 | P01031 | 727 | 0.335570286 | 2.01E−20 | 4.06E−18 | 0.329685453 | 1.09E−19 | 6.23E−19 |
| MIS | AMH | P03971 | 268 | 0.254798925 | 7.34E−12 | 2.12E−10 | −0.473101458 | 2.66E−44 | 2.10E−43 |

TABLE 9
Overlap between plasma proteins which correlate with both clusterin and Dkk4

| | | | | CLU | | | Dkk.4 | | |
|---|---|---|---|---|---|---|---|---|---|
| Protein | GeneName | Uniprot | Entrez | Spearmans | P.value | BH.MTC | Spearmans | P.value | BH.MTC |
| C5b..6.Complex | C5 | P01031 | 727 | 0.335570286 | 2.01E−20 | 4.06E−18 | −0.299845950 | 3.03E−16 | 1.50E−15 |
| MIS | AMH | P03971 | 268 | 0.254798925 | 7.34E−12 | 2.12E−10 | −0.433132124 | 8.03E−36 | 5.97E−35 |

TABLE 10
Overlap between plasma proteins which correlate with both clusterin and DkkL1 (soggy1)

| | | | | CLU | | | Soggy.1 | | |
|---|---|---|---|---|---|---|---|---|---|
| Protein | GeneName | Uniprot | Entrez | Spearmans | P.value | BH.MTC | Spearmans | P.value | BH.MTC |
| ADAMTS.5 | ADAMTS5 | Q9UNA0 | 11096 | 0.255878329 | 5.90E−12 | 1.87E−10 | 0.396409794 | 3.03E−29 | 1.70E−27 |
| Apo.D | APOD | P05090 | 347 | 0.290908515 | 2.67E−15 | 2.71E−13 | 0.504665526 | 3.60E−52 | 1.82E−49 |
| B7 | CD80 | P33681 | 941 | 0.265097484 | 8.79E−13 | 3.17E−11 | 0.279094593 | 4.13E−14 | 3.76E−13 |
| BMP.14 | GDF5 | P43026 | 8200 | 0.272736920 | 1.70E−13 | 8.59E−12 | 0.314163363 | 7.71E−18 | 1.10E−16 |
| C1QBP | C1QBP | Q07021 | 708 | 0.442040252 | 1.39E−37 | 1.41E−34 | 0.276935737 | 6.71E−14 | 5.90E−13 |
| Carbonic.Anhydrase.IV | CA4 | P22748 | 762 | 0.287444818 | 6.05E−15 | 4.71E−13 | 0.427921599 | 8.00E−35 | 8.09E−33 |
| CD22 | CD22 | P20273 | 933 | 0.265509211 | 8.05E−13 | 3.02E−11 | 0.292222627 | 1.98E−15 | 2.10E−14 |
| CD97 | CD97 | P48980 | 976 | 0.269148873 | 3.70E−13 | 1.70E−11 | 0.363303252 | 3.72E−24 | 1.21E−22 |
| CLC4K | CD207 | Q9UJ71 | 50469 | 0.267612428 | 5.15E−13 | 2.10E−11 | 0.281348552 | 2.40E−15 | 2.56E−14 |
| CTACK | CCL27 | Q9Y4X3 | 10850 | 0.267186368 | 5.64E−13 | 2.19E−11 | 0.257958654 | 3.87E−12 | 2.56E−11 |
| Desmoglein.1 | DSG1 | Q02413 | 1828 | 0.276799323 | 6.92E−14 | 3.89E−12 | 0.363855411 | 3.10E−24 | 1.05E−22 |
| I.309 | CCL1 | P22362 | 6346 | 0.379216143 | 1.64E−26 | 5.54E−24 | 0.374598095 | 8.24E−20 | 3.33E−24 |
| IL.18.Rb | IL18RAP | O95256 | 8807 | 0.255363429 | 6.55E−12 | 1.95E−10 | 0.261797012 | 1.75E−12 | 1.22E−11 |
| IL.34 | IL34 | Q6ZMJ4 | 146433 | 0.322058753 | 9.17E−19 | 1.32E−16 | 0.402834549 | 2.54E−30 | 1.51E−28 |
| Kalikrein.6 | KLK6 | Q92876 | 5653 | 0.282688362 | 1.82E−14 | 1.15E−12 | 0.498679805 | 1.37E−30 | 4.63E−46 |
| LCK | LCK | P06239 | 3932 | 0.259956459 | 2.57E−12 | 8.66E−11 | 0.339532981 | 6.27E−21 | 1.32E−19 |
| LD78.beta | CCL3L3 | P16619 | 6349 | 0.285426102 | 9.70E−15 | 7.00E−13 | 0.404658652 | 1.24E−30 | 8.37E−29 |
| OSM | OSM | P13725 | 5008 | 0.252090734 | 1.26E−11 | 3.54E−10 | 0.440852633 | 2.41E−37 | 2.71E−35 |

TABLE 11
Summary of overlap between plasma proteins which
correlate with both clusterin and a Dkk protein

| | Significant | not | Overlap | Pearson's |
|---|---|---|---|---|
| | correlation | correlated | proteins | correlation |
| CLU | 38 | 973 | | |
| DKK1 | 227 | 784 | 2 | 7.90E−21 |
| Dkk3 | 69 | 942 | 0 | 0.2 |
| Dkk4 | 253 | 758 | 2 | 7.80E−20 |
| DKKL1 | 162 | 649 | 18 | 2.20E−16 |
| (Soggy1) | | | | |

4. The same plasma proteins show overlap between (a) clusterin and Dkk1; and (b) clusterin and Dkk4.

TABLE 12

Correlation with

| | Clusterin | Dkk1 | Dkk4 |
|---|---|---|---|
| Protein | p-value | P-value | P-value |
| Complement C5 | 2.00E−20 | 1.09E−19 | 3.03E−16 |
| AntiMullerian hormone | 7.43E−12 | 2.66E−44 | 8.03E−36 |

5. Pathway proteins show correlation with clinical indicators of pathology. Clusterin, Dkk1 and Dkk4 show significant association with rate of decline in cognition (p=7.2×10-6, <0.009, <0.009) and Dkk1 is associated with rate of conversion from MCI to AD (p=0.03).

### CONCLUSIONS

The results shown above demonstrate that levels of clusterin and Dkk proteins in blood correlate with each other and with cognitive decline associated with Alzheimer's disease. This confirms that the molecular pathway that is responsible for the amyloid cascade is detectable in peripheral fluids. Dkk proteins can therefore be used as biomarkers for monitoring the progression of cognitive decline. As the amyloid cascade is a target for anti-Alzheimer's therapy, Dkk proteins can also be used as companion biomarkers for monitoring the efficacy of such treatments.

All publications mentioned in the above specification are herein incorporated by reference. Various modifications and variations of the described embodiments of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for monitoring cognitive decline in a subject, the method comprising:
(i) determining a level of one or more Dickkopf (Dkk) proteins in a blood sample from the subject; and
(ii) comparing the level of the Dkk protein(s) to a reference value;
wherein an increased level of the Dkk protein(s) in the sample compared to the reference value is indicative of increased cognitive decline in the subject.

2. A method according to claim 1, wherein the blood sample comprises blood plasma or serum.

3. A method according to claim 1, wherein the Dkk protein comprises Dkk1, Dkk3, Dkk4 and/or DkkL1 (soggy1).

4. A method according to claim 1, further comprising determining a level of clusterin in the sample, wherein an increased level of clusterin in the sample compared to the reference value is indicative of increased cognitive decline in the subject.

5. A method according to claim 1, further comprising determining a level of one or more additional biomarkers in the sample, wherein the additional biomarker is selected from a plasma protein as defined in any of Tables 3 to 10.

6. A method according to claim 1, wherein the cognitive decline is associated with Alzheimer's disease.

7. A method according to claim 1, wherein the reference value comprises a level of the Dkk protein in a sample from a healthy subject.

8. A method for monitoring the efficacy of a therapeutic treatment for cognitive decline, comprising:
(i) monitoring cognitive decline in the subject by a method as defined in claim 1; and
(ii) repeating the monitoring one or more times following administration of the treatment to the subject;
wherein a decreased level of the Dkk protein(s) in the sample following administration of the treatment is indicative of therapeutic efficacy of the treatment.

9. A method for treating cognitive decline in a subject, the method comprising:
(i) administering a therapeutic treatment for cognitive decline to the subject;
(ii) monitoring cognitive decline in the subject by a method as defined in claim 1, wherein the monitoring is performed before and after administration of the treatment to the subject; and
(iii) providing further treatment to the subject based on the results of the monitoring.

10. A method according to claim 9, wherein if the monitoring indicates a decreased level of the Dkk protein(s) in the sample following administration of the treatment, the further treatment comprises continuing the therapeutic treatment defined in step (i).

11. A method according to claim 9, wherein if the monitoring indicates an increased level of the Dkk protein(s) in the sample following administration of the treatment, the further treatment comprises (a) increasing a dose of the therapeutic treatment defined in step (i); and/or (b) administering an alternative therapeutic treatment to the subject, wherein the alternative therapeutic treatment is different to the therapeutic treatment defined in step (i).

12. A therapeutic agent for use in treating cognitive decline in a subject, wherein the subject has been monitored for cognitive decline by a method as defined in any claim 1.

13. A kit for monitoring cognitive decline in a subject, the kit comprising one or more reagents suitable for detecting one or more Dickkopf (Dkk) proteins in a blood sample from the subject.

14. A kit according to claim 14, wherein the kit comprises one or more antibodies which bind to one or more Dkk proteins.

15. A kit according to claim 13, wherein the kit comprises an ELISA assay for one or more Dkk proteins.
